(19) United States
(12) Patent Application Publication
(10) Pub. No.: US 2019/0307741 A1
(43) Pub. Date: Oct. 10, 2019

(54) VINFLUNINE AND PD1 AND/OR PDL1 INHIBITOR AS PHARMACEUTICAL COMBINATION

(71) Applicant: PIERRE FABRE MEDICAMENT, Boulogne-Billancourt (FR)

(72) Inventors: Anna KRUCZYNSKI, Pompertuzat (FR); Isabelle VANDENBERGHE, Bertre (FR); Pierre FERRE, Toulouse (FR)

(73) Assignee: PIERRE FABRE MEDICAMENT, Boulogne-Billancourt (FR)

(21) Appl. No.: 16/315,275

(22) PCT Filed: Jul. 6, 2017
(86) PCT No.: PCT/EP2017/067012

§ 371 Date: Jan. 4, 2019

(30) Foreign Application Priority Data
Jul. 6, 2016 (EP) 16305851.4

(51) Int. Cl. A61K 31/475; A61K 39/395; A61K 9/00; A61P 35/00

(52) U.S. Cl. CPC A61K 31/475; A61K 39/39558; A61K 2039/505; A61P 35/00; A61K 9/0019

(57) ABSTRACT

The present invention relates to a pharmaceutical combination comprising: (a) vinflunine or a pharmaceutically acceptable salt thereof, and (b) at least one PD1-inhibitor and/or at least one PD-L1-inhibitor or pharmaceutically acceptable salt thereof. Such a pharmaceutical combination is useful as a drug, notably in the treatment of a proliferative disease, such as cancer.

The present invention relates to a pharmaceutical combination comprising (a) vinflunine or a pharmaceutically acceptable salt thereof, and (b) at least one Programmed cell death 1 (PD1) and/or Programmed death-ligand 1 (PD-L1) inhibitor or a pharmaceutically acceptable salt thereof, notably an anti-PD1 or anti-PD-L1 antibody; and optionally at least one pharmaceutically acceptable carrier; methods for preparing the pharmaceutical combinations, and the uses of the pharmaceutical combinations in the treatment of proliferative diseases, such as cancer.

Antimitotic alkaloids extracted from Catharantus roseus and derivatives thereof, notably vinflunine and vinorelbine, have been used in anti-cancer chemotherapy for more than 40 years. In Europe, vinflunine is approved as a treatment option for patients with advanced urothelial cancer and is commercialized under the name Javlor®. The properties of vinflunine have also been studied in vivo and the benefit of its use for the treatment of breast cancer has been established (Bennouna et al. (2003), Ann. Oncol., 14:630-637).

Programmed Cell Death-1 (PD-1, also termed as CD279) is a 55 KD receptor protein related to CD28/CTLA4 co-stimulatory/inhibitory receptor family (Blank et al., 2005 Cancer Immunol Immunother 54:307-314). The genes and cDNAs coding for PD-1 were cloned and characterized in mouse and human (Ishida et al., 1992 EMBO J 11:3887-3395; Shinohara et al., 1994 Genomics 23:704-706). The full length PD-1 contains 288 amino acid residues (NCBI accession number: NP_005009). Its extracellular domain consists of amino acid residues 1-167, and the cytoplasmic C-terminal tail comprises residues 191-288, which has two hypothetical immune-regulatory motifs, an immunoreceptor tyrosine-based inhibitory motif (ITIM; Vivier et al., 1997 Immunol Today 18:286-291) and an immunoreceptor tyrosine switch motif (ITSM; Chemnitz et al, 2004 J Immunol 173:945-954).

Programmed death-ligand 1 (PD-L1) is a 40 kDa type 1 transmembrane protein. The binding of PD-L1 to PD-1 transmits an inhibitory signal which notably reduces the proliferation of these CD8+ T cells at the lymph nodes.

Anticancer chemotherapies can entail the combined use of different agents, mainly in order to reduce the toxicity of one of the agents when used alone and in some cases because the combination may induce an increased efficacy as compared to each of the agents considered alone.

In spite of numerous treatment options for patients with cancer, there remain a need for effective and safe therapeutic agents and a need for new combination therapies that can be administered for the effective long-term treatment of cancer.

Provided herein is a pharmaceutical combination comprising (a) vinflunine or a pharmaceutically acceptable salt thereof, (b) at least one Programmed cell death 1 (PD1) and/or Programmed death-ligand 1 (PD-L1) inhibitor or a pharmaceutically acceptable salt thereof, and optionally at least one pharmaceutically acceptable carrier.

Provided herein is a pharmaceutical combination comprising (a) vinflunine or a pharmaceutically acceptable salt thereof, (b) at least one Programmed cell death 1 (PD1) or Programmed death-ligand 1 (PD-L1) inhibitor or a pharmaceutically acceptable salt thereof (preferably at least one Programmed cell death 1 (PD1) inhibitor or a pharmaceutically acceptable salt thereof), and optionally at least one pharmaceutically acceptable carrier.

Provided herein is a pharmaceutical combination comprising (a) vinflunine, or a pharmaceutically acceptable salt thereof, (b) at least one anti-PD1 antibody or anti-PD-L1 antibody (preferably anti-PD1 antibody), and optionally at least one pharmaceutically acceptable carrier.

Provided herein is a pharmaceutical combination comprising (a) vinflunine, or a pharmaceutically acceptable salt thereof, (b) at least one anti-PD1 antibody, and optionally at least one pharmaceutically acceptable carrier, wherein vinflunine and the anti-PD1 antibody produce a synergistic effect.

Also provided herein is a pharmaceutical combination comprising: (a) vinflunine, or a pharmaceutically acceptable salt thereof, and (b) an anti-PD1 or anti-PD-L1 antibody recognizing the same epitope as already known anti-PD1 or PD-L1 antibody, and optionally at least one pharmaceutically acceptable carrier.

In one embodiment the already known anti-PD1 antibody is selected from the group comprising or consisting in pembrolizumab (anti-PD1), nivolumab (anti-PD1) and lambrozilumab (anti-PD1).

In one embodiment the already known anti-PD-L1 antibody is selected from the group comprising or consisting in avelumab (anti-PDL1), atezolizumab (anti-PDL1) and durvalumab (anti-PDL1), notably avelumab.

Also provided herein is a pharmaceutical combination comprising: (a) vinflunine ditartrate, and (b) an anti-PD1 antibody or anti-PD-L1 antibody notably selected from the list consisting in pembrolizumab (anti-PD1), nivolumab (anti-PD1), lambrozilumab (anti-PD1), atezolizumab (anti-PDL1), durvalumab (anti-PDL1) and avelumab (anti-PDL1), and optionally at least one pharmaceutically acceptable carrier.

Also provided herein is a pharmaceutical combination comprising: (a) vinflunine ditartrate, and (b) pembrolizumab as anti-PD1 antibody, and optionally at least one pharmaceutically acceptable carrier.

In one embodiment, vinflunine and the anti-PD1 or anti-PDL1 antibody are formulated as separate unit dosages for simultaneous, separate or sequential administration.

Also provided are methods of treating a proliferative disease, comprising administering to a patient in need thereof a therapeutically effective amount of a pharmaceutical combination described herein. Notably this pharmaceutical composition will elicit a synergistic anti-tumor effect.

Also provided is a pharmaceutical combination described herein for use in the treatment of a proliferative disease.

In particular the pharmaceutical combination for use in the treatment of a proliferative disease is administered to a subject in such a way that vinflunine and the anti-PD1 antibody produce a synergistic anti-tumor effect.

Also provided is a commercial package comprising as therapeutic agents a pharmaceutical combination of the invention, notably a commercial package comprising (a) vinflunine or a pharmaceutically acceptable salt thereof, (b) at least one Programmed cell death 1 (PD1) and/or at least one Programmed death-ligand 1 (PD-L1) inhibitor or a pharmaceutically acceptable salt thereof, and optionally at least one pharmaceutically acceptable carrier, together with instructions for simultaneous, separate or sequential administration thereof for use in the treatment of a proliferative disease.

Pharmaceutical combinations of the present invention include vinflunine. Vinflunine is a compound of Formula I (20′,20′-difluoro-3′,4′-dihydrovinorelbine) whose formula is described in EP 710 240):

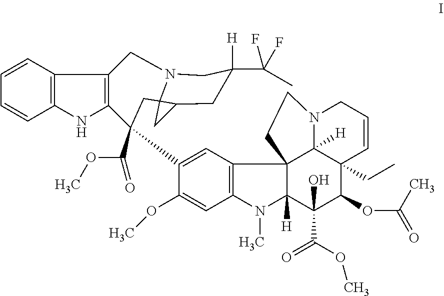

Vinflunine can be produced by any methods known in the art and notably those described in EP 710 240.

Pharmaceutical compositions of the present invention also include a PD1 inhibitor or a PD-L1 inhibitor.

In one embodiment, the PD1 inhibitor is a monoclonal antibody, notably selected in the group comprising or consisting in pembrolizumab (anti-PD1), nivolumab (anti-PD1) and lambrozilumab (anti-PD1), notably pembrolizumab.

In one embodiment, the PD1-L1 inhibitor is a monoclonal antibody, notably selected in the group comprising or consisting in avelumab (anti-PDL1), atezolizumab (anti-PDL1) and durvalumab (anti-PDL1), notably avelumab.

The term “anti-PD1 antibody” should be interpreted as similar to “PD1 antibody” and is defined herein to refer to an antibody or an antigen binding fragment thereof able to bind to PD1 (PD1 antagonist) notably located on lymphocytes. Preferably, the anti-PD1 antibody competes for the ligand receptor binding notably by occluding the ligand-binding region.

The term “anti-PD-L1 antibody” should be interpreted as similar to “PD-L1 antibody” and is defined herein to refer to an antibody or an antigen binding fragment thereof able to bind to PD-L1 (PD-L1 antagonist). Preferably, the anti-PD-L1 antibody inhibits the fixation of PD-L1 to PD1 notably by occluding the receptor-binding region.

According to the invention, the term “salt” is understood to be a salt of vinflunine or of a PD1 or PD-L1 inhibitor that can be present alone or in mixture with the free base of the compound. Such salts are formed, for example, as acid addition salts by reacting the free base form of the compound with a pharmaceutically acceptable inorganic or organic acid. The acids that may be used to prepare pharmaceutically acceptable acid addition salts of such basic compounds of the present invention are those that form non-toxic acid addition salts, i.e., salts containing pharmaceutically acceptable anions, such as the acetate, benzoate, bromide, chloride, citrate, fumarate, hydrobromide, hydrochloride, iodide, lactate, maleate, mandelate, nitrate, oxalate, salicylate, succinate, ditartrate and tartrate salts. The salts according to the invention are preferably pharmaceutically acceptable salts. In one embodiment the vinflunine's salt is tartrate or ditartrate, in particular ditartrate.

The term “combination” or “pharmaceutical combination” is defined herein to refer to either a fixed combination in one dosage unit form, a non-fixed combination or a kit of parts for the combined administration where vinflunine or a pharmaceutically acceptable salt thereof and the PD1 and/or PD-L1 inhibitor or a pharmaceutically acceptable salt thereof may be administered simultaneously or independently at the same time or separately within time intervals. In one embodiment, the combination partners (i.e., vinflunine and PD1 and/or PD-L1 inhibitor) are administered simultaneously, independently at the same time, or separately within time intervals that allow that the combination partners to show a cooperative, e.g., synergistic/joint therapeutic effect.

The term “pharmaceutical composition” is defined herein to refer to a mixture or solution containing at least one therapeutic agent to be administered to a subject, e.g., a mammal or human, in order to treat a particular disease or condition affecting the mammal.

The term “pharmaceutically acceptable” is defined herein to refer to those compounds, materials, compositions and/or dosage forms, which are, within the scope of sound medical judgment, suitable for contact with the tissues of a subject, e.g., a mammal or human, without excessive toxicity, irritation, allergic response and other problem complications commensurate with a reasonable benefit/risk ratio.

The term “co-administration” or “combined administration” as used herein is defined to encompass the administration of the selected therapeutic agents to a single patient, and are intended to include treatment regimens in which the agents are not necessarily administered by the same route of administration or at the same time.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present invention, the term “treat” also denotes to arrest, delay the onset (i.e., the period prior to clinical manifestation of a disease) and/or reduce the risk of developing or worsening a disease.

The term “joint therapeutic effect” as used herein means that the therapeutic agents may be given to the patient simultaneously or separately (e.g., in a chronologically staggered manner, for example a sequence-specific manner) in such time intervals that they show an interaction (e.g., a joint therapeutic effect, for example a synergistic effect). Whether this is the case can, inter alia, be determined by following the blood levels and showing that both compounds are present in the blood of the human to be treated at least during certain time intervals.

The term “pharmaceutically effective amount” or “clinically effective amount” of a combination of therapeutic agents is an amount sufficient to provide an observable improvement over the baseline clinically observable signs and symptoms of the disorder treated with the combination.

The terms “antibody” and “antibodies” are used interchangeably in the broadest sense and include monoclonal antibodies, (e.g., full length or intact monoclonal antibodies) and polyclonal antibodies, which can be multivalent antibodies or multispecific antibodies (e.g., bispecific antibodies). In another aspect, the antibody can be an isolated, engineered, chemically synthesized, or recombinant antibody. They can be in the form of antibody fragments such as antigen-binding fragments, so long as they exhibit the desired biological activity. In one embodiment, the antibody is a recombinant antibody. Preferably it is a monoclonal antibody. In another aspect, the antibody of the invention can be a chimeric or humanized antibody. In yet another aspect, said antibody is a fully human antibody.

By “binding fragment” or “antigen-binding fragment” of an antibody, it is intended to indicate any peptide, polypeptide, or protein retaining the ability to bind to the target (also generally referred as antigen) of the antibody. In an embodiment, such “antigen binding fragments” are selected in the group consisting of Fv, scFv (sc for single chain), Fab, F(ab′)2, Fab′, scFv-Fc fragments or diabodies, or any fragment of which the half-life time would have been increased by chemical modification, such as the addition of poly(alkylene) glycol such as poly(ethylene) glycol (“PEGylation”) (pegylated fragments called Fv-PEG, scFv-PEG, Fab-PEG, F(ab′)₂-PEG or Fab′-PEG) (“PEG” for Poly(Ethylene) Glycol), or by incorporation into a liposome, said fragments having at least one of the characteristic CDRs of the antibody according to the invention. In one embodiment, the “antigen binding fragments” will be constituted or will comprise a partial sequence of the heavy or light variable chain of the antibody from which they are derived, said partial sequence being sufficient to retain the same specificity of binding as the antibody from which it is descended and a sufficient affinity, for example at least equal to 1/100, as a further example to at least equal to 1/10, of the affinity of the antibody from which it is descended, with respect to the target.

As used herein, the terms “subject”, “individual,” or “patient,” used interchangeably, refer to any animal, including mammals such as humans, primates, mice, rats, other rodents, rabbits, dogs, cats, swine, cattle, sheep and horses. In some embodiments, the subject is a human.

The term “synergistic effect” as used herein refers to action of two therapeutic agents such as, for example, vinflunine and the anti-PD1 antibody producing an effect, for example, slowing the symptomatic progression of a proliferative disease, particularly cancer, or symptoms thereof, which is greater than the simple addition of the effects of each therapeutic drug administered by themselves. A synergistic effect can be calculated notably by methods known in the state of the art. This unexpected synergistic reaction may allow reduction in the dose required for each compound, leading to a reduction in the side effects and enhancement of the long-term clinical effectiveness of the compounds in treatment. The synergistic anti-tumor effect can notably be evaluated on the basis of the evaluation of the progression of the cancer in an individual compare to the mean of the evolution of the cancer in individual treated with only one the each compounds.

Also provided is the use of a pharmaceutical composition according to the invention, in the treatment of a proliferative disease.

In one embodiment, the proliferative disease according to the invention is cancer. Examples of cancers which may be treated with a combination of the invention include bladder cancer, breast cancer, colorectal cancer (CRC) (including metastatic colorectal cancer), melanoma, lung cancer (including non-small cell lung cancer (NSCLC)), kidney cancer such as e.g., renal cell carcinoma (RCC), liver cancer, endometrial cancer, acute myelogenous leukemia (AML), myelodysplasia syndromes (MDS), thyroid cancer, particularly papillary thyroid cancer, pancreatic cancer, neurofibromatosis or hepatocellular carcinoma.

In one embodiment, a method of treating a proliferative disease may comprise (i) administration of vinflunine in free or pharmaceutically acceptable salt form, (ii) administration of an anti-PD1 antibody and/or an anti-PD-L1-antibody, simultaneously or sequentially in any order, in jointly therapeutically effective amounts, (for example in synergistically effective amounts), e.g. in daily or intermittently dosages corresponding to the amounts described herein.

The effective dosage of each of the combination partners employed in the combination of the invention may vary depending on the particular compound or pharmaceutical composition employed, the mode of administration, the condition being treated, and the severity of the condition being treated. Thus, the dosage regimen of the combination of the invention is selected in accordance with a variety of factors including the route of administration and the renal and hepatic function of the patient. In some embodiments the unit dosage forms containing the combination of agents as described herein will contain the amounts (or less) of each agent of the combination that are typically administered when the agents are administered alone.

Frequency of dosage may vary depending on the compound used and the particular condition to be treated. In general, the use of the minimum dosage that is sufficient to provide effective therapy is preferred. Patients may generally be monitored for therapeutic effectiveness using assays suitable for the condition being treated, which will be familiar to those of ordinary skill in the art.

The effective dosage of each of the combination partners may require more frequent administration of one of the compound(s) as compared to the other compound(s) in the combination. Therefore, to permit appropriate dosing, in one embodiment packaged pharmaceutical products may contain one or more dosage forms that contain the combination of compounds, and one or more dosage forms that contain one compound of the combination of compounds, but not the other compound(s) of the combination. In yet another embodiment, packaged pharmaceutical products may contain one or more dosage forms of one, two and/or three of each of the combination partners.

When the combination partners employed in the combination of the invention are applied in the form as marketed as single drugs, their dosage and mode of administration may be in accordance with the information provided on the package insert of the respective marketed drugs, if not mentioned herein otherwise.

Administration of the compositions may be oral, intravenous, respiratory (e.g., nasal or intrabronchial), parenteral (besides i.v., such as intraperitoneal and subcutaneous injections), transdermal (including all administration across the surface of the body). Preferably, the administration of the compositions according to the combination of the present invention is done intravenously. While the components of the invention may be delivered via the same route, a product or pack according to the invention may contain vinflunine or a pharmaceutically acceptable salt thereof, for delivery via a different route than that of the anti-PD1 antibody and/or anti-PD-L1-antibody, e.g., one component may be delivered orally, while the other is administered intravenously. In another embodiment, vinflunine or a pharmaceutically acceptable salt thereof and the anti-PD1 (notably pembrolizumab) or anti-PD-L1 are both delivered via the same route, e.g., i.v. Other variations would be apparent to one skilled in the art and are contemplated within the scope of the invention. Preferred pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. In one embodiment, preferred injectable compositions for vinflunine or a pharmaceutically acceptable salt thereof are described in WO2005070425.

Vinflunine or a pharmaceutically acceptable salt thereof may be administered to a suitable subject in single or divided doses at an effective dosage in the range of between 200 and 400 mg/m², notably between 280 and 320 mg/m² every three weeks. In on embodiment vinflunine or a pharmaceutically acceptable salt thereof is administered intravenously or orally, notably intravenously. Notably vinflunine or a pharmaceutically acceptable salt thereof is formulated in a sterile aqueous solution at a pH of between 3 and 4. Notably, vinflunine, in the form of its ditartrate salt, is administered under the brand name Javlor®. The dose regimen regarding vinflunine or a pharmaceutically acceptable salt thereof is therefore between 250 mg/m² and 320 mg/m², preferably about 320 mg/m² on Day 1, as a loading dosage for 3 weeks. This administered dosage may be followed by the same dosage on day 22, thus making the starting of a new cycle; Day 22 being the Day 1 of the second treatment cycle. Such 3 weeks cycle with one administration of vinflunine or a pharmaceutically acceptable salt thereof on Day 1 at the dosage of 250 mg/m² to about 320 mg/m², preferably 320 mg/m² may be repeated from about 3 to 20 times, preferably for about 3 to 19 times, even preferably for about 8 times.

The anti-PD1 antibody may be administered daily to a suitable subject in single or divided doses at an effective dosage in the range of about 0.001 to about 100 mg per kg body weight per day, for example about 1 mg/kg/day to about 35 mg/kg/day, in single or divided doses (for example 2 mg/kg three times in a week). In one embodiment the anti-PD1 antibody is administered intravenously.

### LEGEND OF THE FIGURE

FIG. 1: Survival curves in survival percentage in function of time (time post tumor induction) in days.

### EXAMPLE

The antitumour activity of vinflunine alone or in combination with an anti-PD1 antibody was evaluated in C3H/HEJ mice bearing orthotopic MBT-2 mouse bladder tumors.

### Materials and Methods

### 1. Products

Vinflunine ditartrate is administered to mice at a concentration of 1.2 mg/ml in a saline buffer (0.9% NaCl).

The anti-PD1 antibody (clone: RMP1-14, catalog: BE0146, B/N: 5311-10/0914B, Bioxcell), is administered to mice at a concentration of 1 mg/ml in PBS. This antibody was notably described in J Immunol. 2005 Aug. 1; 175(3):1586-92.

Synergistic antitumor should be obtained regardless of the anti-PD1/PDL1 antibody clones used. In fact, the response of tumor to checkpoint blockade is more closely associated with inherent tumor immunogenicity and immune cell infiltration than with the tissue origin or antibody origin. The proof of the efficacy of an anti-PD1 antibody is always made on Mouse model with antibodies which recognize the mouse PD1 (and not the human PD1). The experiments on mouse model are considered by the scientific community as highly predictive of what would be obtained in human. For example, Selby et al (PlosOne 2016 DOI:10.1371) demonstrated a synergistic antitumor activity on CT26 and MC38 models using the 4H2 anti-mouse PD1 whereas Sivan et al (Science 2015 350 :1084-1089) confirmed these results using anti-mouse PDL1 antibody. Charles River CRO also published synergistic activities in various tumor models using the RMP1-14 anti-mouse PD1 clone (http://www.criver.com/files/pdfs/discovery/immune-cells-syngeneic-models.aspx).

From these concordant studies, it is proved that the results obtained with the anti-mouse PD1 RMP1-14 antibody could be reproduced with other anti-mouse PD1 clones and also be translated in clinical settings with anti-human PD1.

### Treatment Doses:

Vinflunine ditartrate has been injected at 12 mg/kg,

The anti-PD1 antibody has been injected at 10 mg/kg/inj.

Vinflunine ditartrate and the anti-PD1 antibody have been administered at a dose volume of 10 ml/kg/adm (i.e for one mouse weighing 20 g, 200 μl has been administered).

Vinflunine ditartrate has been injected intraperitoneally (IP) into the peritoneal cavity of mice.

The anti-PD-1 antibody has been injected intraperitoneally (IP) into the peritoneal cavity of mice.

### 2. Cancer Cell Line and Culture Conditions

The murine MBT-2 cell line was derived from a carcinogen-induced bladder tumor in C3H/HeJ mice (Soloway et al. Surg Forum. 1973; 24: 542-544).

Tumor cells have been e grown as monolayer at 37° C. in a humidified atmosphere (5% CO2, 95% air). The culture medium was RPMI 1640 containing 2 mM L-glutamine (supplemented with 10% fetal bovine serum). The cells are adherent to plastic flasks. For experimental use, tumor cells have been detached from the culture flask by a 5 minute treatment with trypsin-versene, in Hanks' medium without calcium or magnesium and neutralized by addition of complete culture medium. The cells have been counted in a hemocytometer and their viability has been assessed by 0.25% trypan blue exclusion assay. Two cell preparations have been prepared in order to graft.

### 3. Animals

Eighty one (81) healthy female C3H/HeJ (C3H/HeOuJ) mice were obtained from The Jackson Laboratory (Bar Harbor, Me.).

Antitumour activity of vinflunine ditartrate alone, anti-PD1 antibody alone or of the combination was evaluated against MBT-2 cells orthotopically implanted by instillation C3H/HEJ mice.

Induction of Orthotopic MBT 2 Tumors in Animals:

A 4-hour water deprivation period has been observed just prior starting the tumor induction procedure. Eighty one mice have been anaesthetized using Ketamine and Xylazine cocktail and have been positioned upside-down (to reduce as much as possible damages to the urethra and ureters). A catheter has been inserted in the bladder through the urethra and 30 μL 0.1 N HCl has been injected. HCl has been allowed to remain in the bladder for 15 seconds and has been then replaced by 30 μL 0.1N NaOH which has been allowed to remain in the bladder for 15 seconds. Then the bladder has been drained and flushed with PBS 1× pH7.4. After flushing the bladder, MBT-2 tumor cells (2×106 cells in 50 μL RPMI1640 medium) have been instilled into the bladder and allowed to remain for 45 minutes. At the end of this 45 minute period, the bladder has been drained. A subcutaneous injection of 0.9% NaCl solution has been performed at the end of MBT-2 tumor cells instillation. The day of tumor induction has been considered as the day 0.

Treatment Schedule:

The treatment has started at D3. Mice have been distributed according to their individual body weight into 6 groups each of 13 animals using Vivo Manager® software (Biosystemes, Couternon, France). A statistical test (analysis of variance) has been performed to test homogeneity between groups. The treatment schedule is as follows:

The animals from group 1 have received 2 cycles of 3 IP injections of vinflunine vehicle (saline) every 2 days. The cycles have been separated by a two days period of wash out (At D3, D5, D7, D10, D12 and D14: (Q2D×3)×2W).

The animals from group 2 have received 2 cycles of 3 IP injections of vinflunine ditartrate at dose 12 mg/kg every 2 days. The cycles have been separated by a two days period of wash out (At D3, D5, D7, D10, D12 and D14: (Q2D×3)×2W).

The animals from group 3 have received one IP injection of anti-PD1 antibody at 10 mg/kg/inj twice weekly for two consecutive weeks (at D3, D6, D10 and D13: TW×2).

The animals from group 4 have received 2 cycles of 3 IP injections of vinflunine ditartrate at dose 12 mg/kg every 2 days (the cycles have been separated by a two days period of wash out (At D3, D5, D7, D10, D12 and D14: (Q2D×3)×2W) in combination with one IP injection of anti-PD1 antibody at 10 mg/kg/inj twice weekly for two consecutive weeks (at D3, D6, D10 and D13: TW×2). The day of concomitant injection (e.g. D3 or D10), the injection of anti-PD1 antibody has been performed 15 minutes before vinflunine ditartrate injection.

At D30, or when mice in vehicle treated group have presented clinical sign of tumour development, all mice have been terminated, mice have been individually terminated when mice have presented clinical sign of tumour development. At the time of termination, bladder from each surviving mouse has been collected and weighed. Samples have then been fixed in 4% neutral buffered formalin for 24 to 48 h, and then embedded in paraffin (Histosec®, Merck, Darmstadt, Germany). According to bladder and tumour size, if necessary, sample has been cut in two equivalent parts.

### Results:

The following results were obtained (Data relative to vehicle) (Table 1):

TABLE 1

| | | ILS: Increase of Life |
|---|---|---|
| | Survival gain (compared to | Span (compared to |
| Group | vehicle) Median (Days) | vehicle) (%) |
| Group 1 | 0 | 0 |
| (Vehicle) | | |
| Group 2 | 10 | 46 |
| (vinflunine) | | |
| Group 3 (anti- | 12 | 55 |
| PD1) | | |
| Group 4 | >40 | 182 |
| (vinflunine + | | |
| anti-PD1) | | |

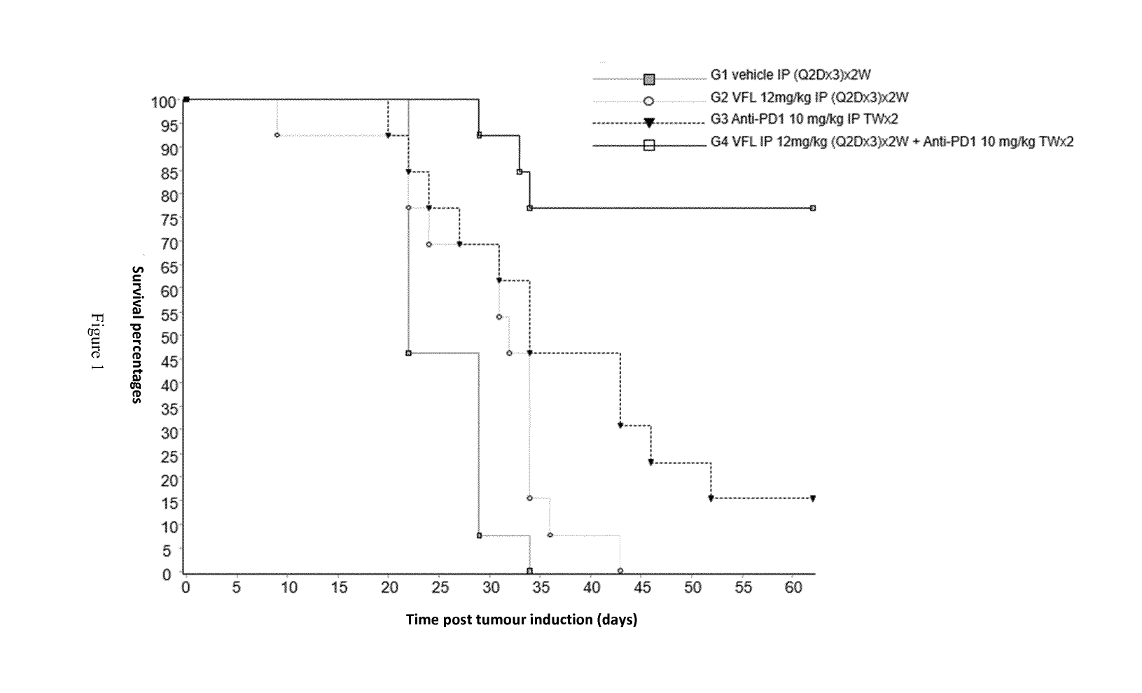

The survival curves are represented in FIG. 1.

As shown in Table 1 and FIG. 1 the combination of vinflunine with an anti-PD1 antibody increases in a synergistic manner the survival and the ILS (ILS=T/C−100, C=median day of death of the control group, T=median day of death of the treated group) of the animals.

1. A pharmaceutical combination comprising:
(a) vinflunine or a pharmaceutically acceptable salt thereof, and
(b) at least one PD1-inhibitor and/or at least one PD-L1-inhibitor or a pharmaceutically acceptable salt thereof.

2. The pharmaceutical combination of claim 1, comprising
(a) vinflunine or a pharmaceutically acceptable salt thereof, and
(b) at least one PD1-antibody or an antigen binding fragment thereof.

3. The pharmaceutical combination of claim 2, wherein the PD1 antibody is selected from the group consisting of pembrolizumab, nivolumab, and lambrozilumab.

4. The pharmaceutical combination of claim 3, wherein the PD1 antibody is pembrolizumab.

5. The pharmaceutical combination according to claim 1, wherein said vinflunine pharmaceutically acceptable salt is vinflunine ditartrate.

6. The pharmaceutical combination according to claim 1, further comprising at least one pharmaceutically acceptable carrier.

7. The pharmaceutical combination according to claim 1, wherein the vinflunine or a pharmaceutically acceptable salt thereof and the anti-PD1 inhibitor and/or anti-PD-L1 inhibitor or a pharmaceutically acceptable salt thereof are formulated as single unit dosage forms for simultaneous, separate or sequential administration.

8. The pharmaceutical combination according to claim 1, wherein the vinflunine or a pharmaceutically acceptable salt thereof is formulated for intravenous administration.

9. The pharmaceutical combination according to claim 1, wherein the anti-PD1 inhibitor and/or anti-PD-L1 inhibitor or a pharmaceutically acceptable salt thereof is formulated for IV administration.

10-13. (canceled)

14. A method of treating a proliferative disease comprising administering to a patient in need thereof a therapeutically effective amount of a pharmaceutical combination according to claim 1.

15. The method according to claim 14, wherein the proliferative disease is cancer.

16. The method according to claim 15, wherein the cancer is selected from bladder cancer, breast cancer, colorectal cancer (CRC), melanoma, lung cancer, kidney cancer, liver cancer, endometrial cancer, acute myelogenous leukemia (AML), myelodysplasia syndromes (MDS), thyroid cancer, pancreatic cancer, neurofibromatosis and hepatocellular carcinoma.

17. The method according to claim 16, wherein the colorectal cancer (CRC) is metastatic colorectal cancer; the lung cancer is non-small cell lung cancer (NSCLC); the kidney cancer is renal cell carcinoma (RCC); the thyroid cancer is papillary thyroid cancer.

18. The method according to claim 16, wherein the cancer is bladder cancer or breast cancer.
